(19) United States
(12) Patent Application Publication
(10) Pub. No.: US 2013/0184517 A1
(43) Pub. Date: Jul. 18, 2013

(54) System and Method for Measuring and Controlling Stress

(71) Applicant: Ronda Collier, Los Gatos, CA (US)

(72) Inventor: Ronda Collier, Los Gatos, CA (US)

(21) Appl. No.: 13/544,929

(22) Filed: Jul. 9, 2012

Related U.S. Application Data
(60) Provisional application No. 61/505,426, filed on Jul. 7, 2011.

(51) Int. Cl. A61M 21/02

(52) U.S. Cl. CPC A61M 21/02
USPC 600/28

(57) ABSTRACT

A computer program product for processing heart rate information signals, which, when run on a computer controls the computer to estimate stress levels of a user in real time and provide generative feedback and alerts to the user when appropriate.

This application claims the benefit of U.S. Provisional Application No. 61/505,426, filed Jul. 7, 2011, which is hereby incorporated by reference.

### BACKGROUND

Today many sophisticated diagnoses can be made from vital signs such as heart beat data via complex mathematical data analysis techniques. Heart beat data from measurement devices such ECG and Plethysomography is used to determine metrics such as heart rate and heart rate variability (HRV), and thus expand the range of these data for insight into health and fitness. HRV is a “view” into the Autonomic Nervous System (ANS). The sympathetic “fight or flight” branch of the nervous system speeds the heart up, while the parasympathetic “rest and digest” branch slows the heart down. The interplay between these two branches of the ANS causes variability in the beat to beat heart rhythm. Because our blood pressure, digestion and respiration as well as our thoughts, emotions, perceptions and environment are tightly coupled with the ANS, much can be revealed by monitoring the ANS activity.

HRV is an established non-invasive and inexpensive method for monitoring the ANS. The standard HRV measures include a variety of time domain, frequency domain and non-linear measures of the heart rate time series. However, while the established HRV metrics provide insight into general health, HRV is limited as a standalone measure, and requires significant processing in order to be a useful tool for individuals to actively manage daily stressors.

Currently there are applications in this field that measure HRV and provide biofeedback in order to effect a specific physiological change such as breathing frequency or depth. Examples of such products are ‘emWave’ by HeartMath, ‘Heart Tracker’ by Biocom Technologies, ‘Journey to Wild Divine’ by Wild Divine and ‘Stress Doctor’ by Azumio. These products are measuring and encouraging a state of “coherence” where the breathing rate and heart rate are in sync and the ANS exhibits a very specific pattern with activity isolated around the 0.1 Hz HRV frequency. In addition there are health assessment systems, such as ‘Heart Rhythm Scanner’ by Biocom Technologies and Nevrokard that measure HRV and provide comprehensive assessment of the ANS.

While the existing HRV products work well for coherence training and ANS assessment, they do not provide real time feedback during regular activities. Many daily stressors are caused by recurring events and thinking patterns, such as heavy traffic and needless worry. Changing our patterns require behavior changes which are notoriously difficult to effect. Thus, there is a need for generative feedback systems and methods, that is real time alerts that bring awareness in the moment and help change unhealthy behaviors.

### SUMMARY

A computer program product that as input receives heart beat information from a heart rate monitor and, when run on a computer, processes the heart beat intervals to detect the state of the Autonomic Nervous system in real time using a re-programmable personalized Neural Network, applies adaptive scaling to HRV parameters to customize the detection for individual, and as output generates immediate feedback that includes measured stress levels and, when appropriate, alerts using audio, visual, alphanumeric displays on the computer platform and audio feedback, that may be part of a stress level-audio feedback loop, to a wearable audio transducer.

### BRIEF DESCRIPTION OF THE DRAWINGS

FIG. 1 is a system level depiction of the overall communication platforms.

FIG. 2 is a flow chart showing the steps for generating training and test vectors for the MLP.

FIG. 3 is a flow chart showing the steps for detecting stress using a Neural

Network Classifier.

FIG. 4 is represents the RR interval filter.

FIG. 5 shows the HRV calculations on a 5 minute window of RR intervals.

FIG. 6 is a detailed depiction of the normalization and sensitivity scaling.

FIG. 7 shows the audio feedback flow.

FIG. 8 shows the HRV calculations including some non-linear HRV parameters.

FIG. 9 shows an alternative ANS detection scheme that includes non-linear HRV parameters.

FIG. 10 illustrates a flow chart for generating test and training vectors that include non-linear HRV parameters.

FIG. 11 shows real life HRV, heart rate and stress levels of an individual working and having their computer hang.

FIG. 12 shows HRV, heart rate and stress levels during an acupuncture session.

FIG. 13 shows the application display of heart rate, HRV and stress levels.

FIG. 14 shows an example of visual representations of stress levels.

FIG. 15 is an exemplary mobile computing device.

### DETAILED DESCRIPTION

Definitions

In the following, we refer to various quantities with abbreviations as follows:

Generative Feedback=Feedback that tracks behavior and also drives behavior

ANS=Autonomic Nervous System

ULF=Ultra low frequency

VLF=very low frequency

LF=low frequency

HF=high frequency

HR=heart rate

HRV=heart rate variability

PSD=Power Spectral Density

rMSSD=Root-mean-square of the successive normal sinus RR interval difference

SDNN=standard deviation of all normal sinus RR intervals

RR Interval=Time duration between two consecutive R waves of the ECG.

Ectopic Beat=An irregular beat arising in the heart due to variations in the hearts electrical conductance system

R wave=The first upward deflection in the ECG waveform

ECG=Electro Cardiogram used to monitor heart electrical activity

EEG=Electroencephalogram measures of brain electrical activity

ApEn=Approximate Entropy which quantifies the regularity of RR intervals

DFA=Detrended Fluctuation Analysis permits detection of self similarity in RR intervals

FD=Fractal dimension is a measure of regularity of RR intervals and quantifies sensitivity to initial conditions.

LLE=Local Lyapunov Exponent is a measure of chaoticity of RR intervals

Poincare' Plot=Graphical representation of short term and long term HRV

Holter Monitor=A portable device for recording heartbeats over a period of 24 hours or more.

System Overview

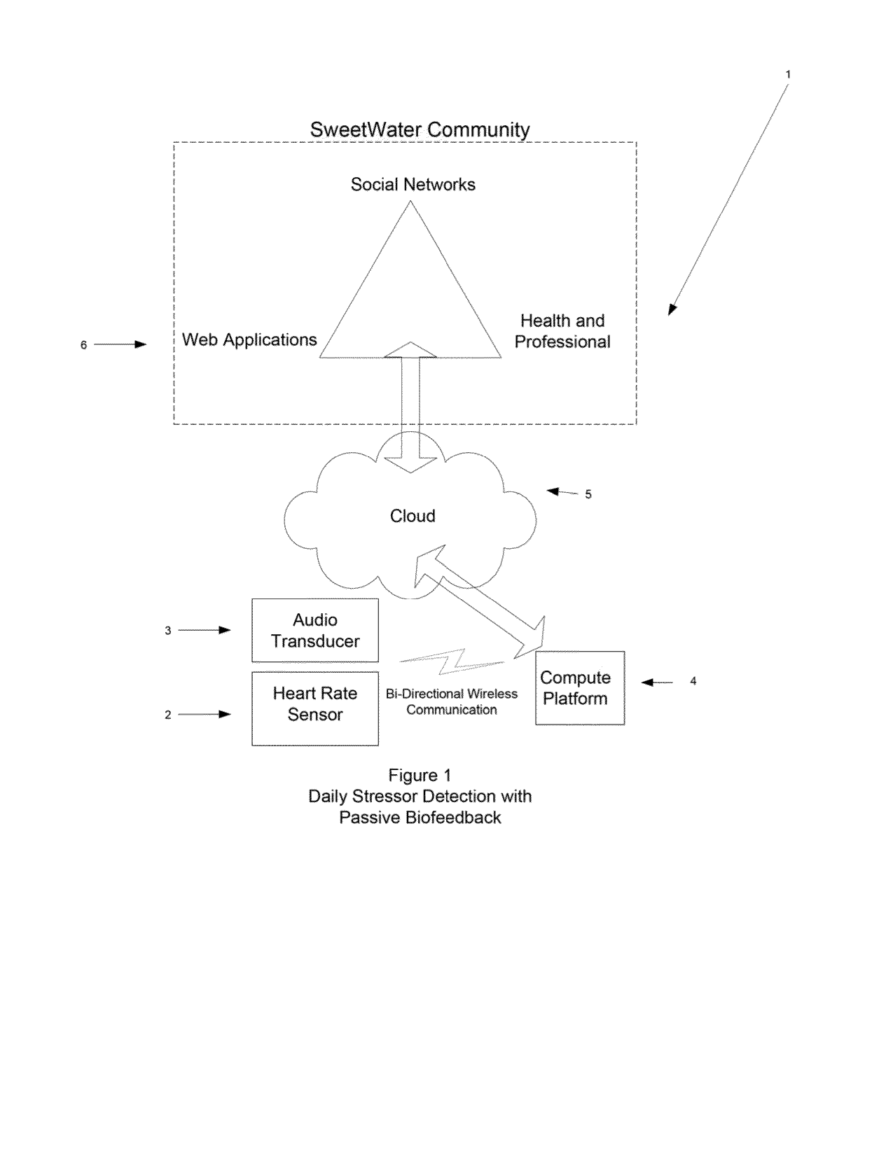

With reference first to FIG. 1, the present disclosure is directed to a system 1 and methods, described further below, for monitoring user heart rate for analysis to detect daily stress that causes imbalance to the Autonomic Nervous System. The present disclosure is directed to analysis of that data on the computer platform or in the cloud, and further to providing ongoing real time feedback and alerts in the form of audio, video, alphanumerical or graphical media. The audio feedback may be transmitted wirelessly to a bone conducting transducer 3 worn by an individual.

Monitoring of heart rate is accomplished via a medical or consumer heart rate measurement apparatus including and not limited to an ECG, Holter Monitor, Pulse Oximeter or other plethysmographic method, chest strap, or clothing incorporated sensor 2. This heart rate data is transmitted via wire or wireless to a computing platform 4 for analysis. The computing platform includes and is not limited to a smart phone, tablet or desktop computer.

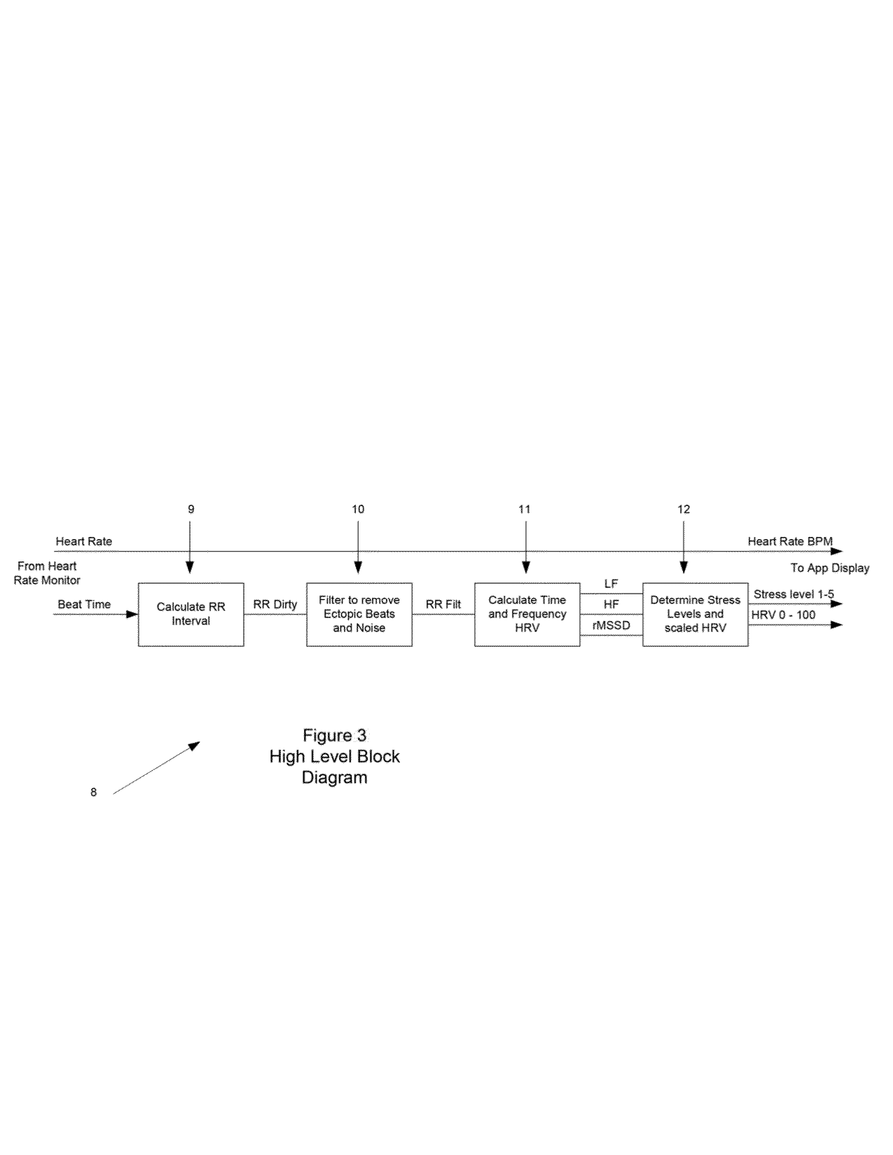

Referring to FIG. 3, the beat to beat or RR intervals are then calculated 9 from the heart rate data if they are not provided directly from the heart rate measurement device. These intervals are filtered 10 and then processed to calculate the corresponding HRV values 11. The heart rate and HRV information are input into Multilayer Perceptron Neural Network 12 to classify the data into one of five stress levels.

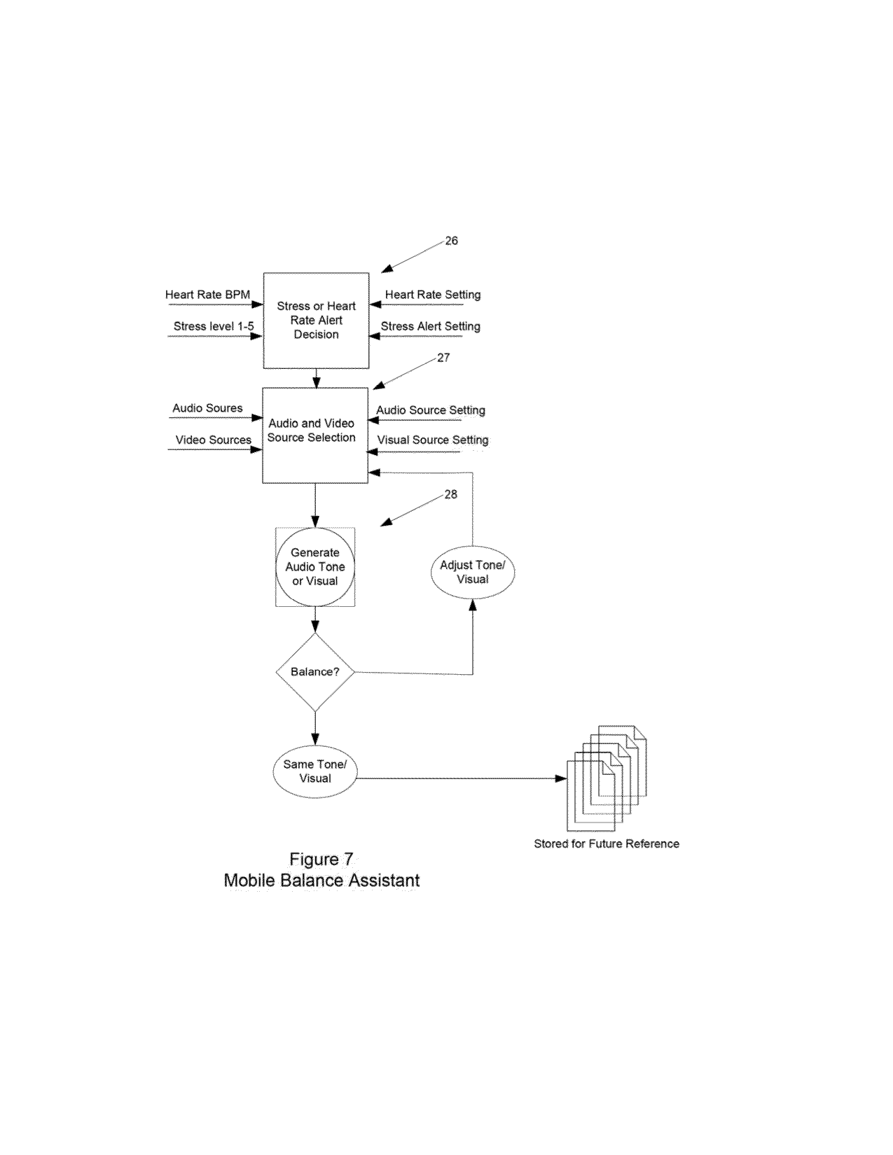

FIG. 7 illustrates the alert detection flow. When a user specified stress level is detected 26 an alert on signal is received by the alert source detection module 27. If the alert is audio, it is sounded from the compute platform or transmitted to the user worn bone transducer. If the alert is visual, it is displayed on the compute platform. As the program continues to detect stress levels, the audio and/or visual alert 28 is adjusted. This adjustment can be a result of a change in stress levels or it can be a result of no change in stress levels with the intention of inducing a lower stress state in the user. This feedback loop consists of tone generation or visual indicator, HRV measurement, tone/visual adjustment and again to tone/visual generation. This iterative process may continue until the desired outcome is achieved. The alert details and associated stress levels are stored for future use.

Referring again to FIG. 1, at the end of a monitoring session, details of the session, including the raw RR intervals are stored and uploaded to the cloud to be used in the web applications. In addition the raw data from an individual, combined with user input, is used to create a custom classifier. The hidden node weights from the custom classifier are then downloaded to the compute platform and a new individually customized stress detection algorithm is used for future monitoring sessions. This process can be repeated indefinitely.

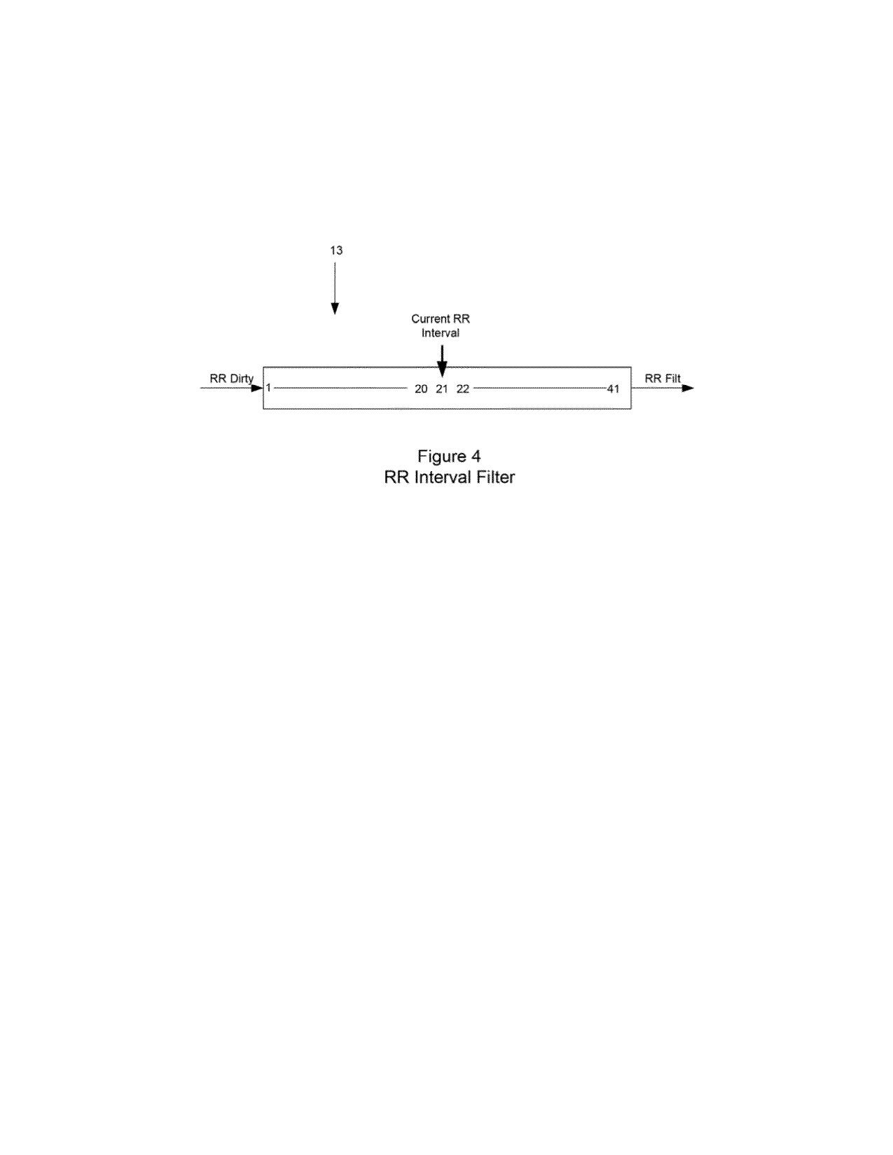

Referring again to FIG. 3, the heart rate monitor may provide the heart beat time or the RR intervals directly. In the event that the beat time is provided, the RR intervals are calculated as RRt=Beat Time (t+1)−Beat Time (t). The RR intervals, whether they were calculated or provided by the heart rate monitor, are then filtered 10 to remove any noise or ectopic beats. FIG. 4 shows the detailed filter 13 that works as follows:

41 RR intervals are queued in a “FIFO” type array

The 21^{st} RR interval is the current intervals

Intervals 1-20 and 22-41 are averaged

If the current interval is +/−20% of the averages of 1-20 and 22-41 then it is considered a normal and labeled “N”.

If the current interval falls outside the +/−20% range it is labeled “O”

If an interval is less than 0.4 seconds it is labeled “I”

If an interval is more than 2 sec it is labeled ‘X”

Only “N” intervals are used for HRV calculation

This is repeated each time a new RR interval is input into the FIFO

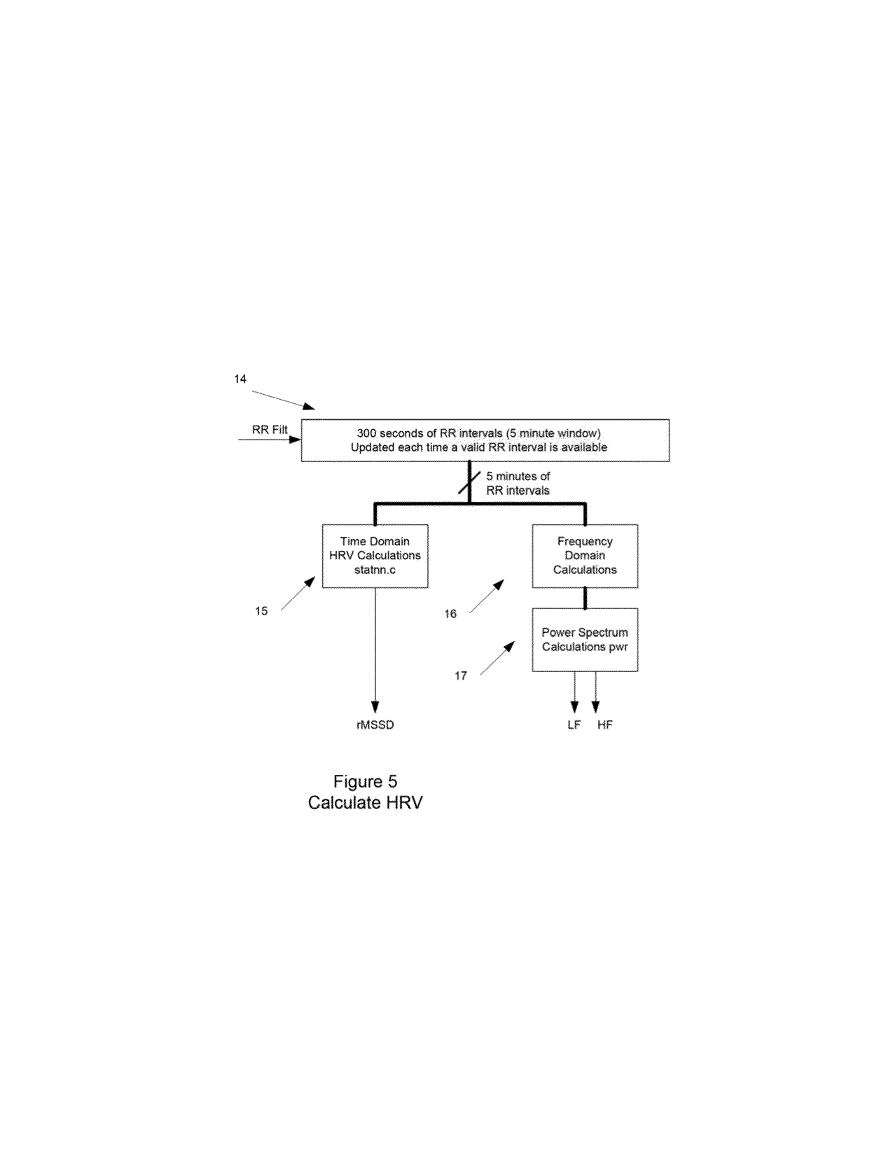

The filtered RR intervals are stored in another “FIFO” type array (FIGS. 5) 14, and 300 seconds worth of RR intervals are collected to create a 5 minute window that is then processed. The time domain HRV calculation block 15 computes and is not limited to rMSSD. The frequency domain HRV calculation block 16 computes and is not limited to LF and HF. The Power Spectral Density (PSD) of the HRV frequency components LF and HF is calculated using the Lomb Periodogram.

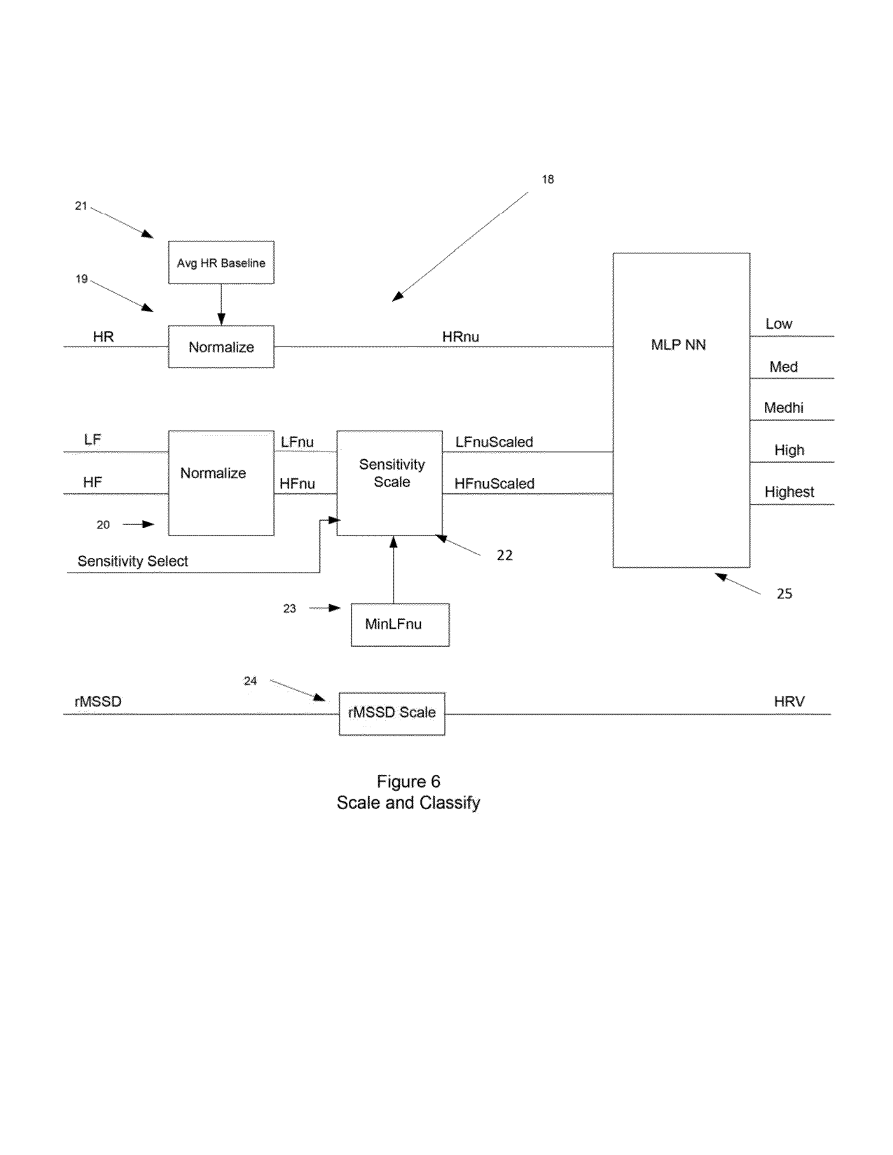

Once the time and frequency HRV parameters are calculated, they are processed to determine the stress level of the individual. FIG. 6 shows one such embodiment of the stress detection process. The heart rate, LF and HF values are normalized 19,20 as follows:

Normalize HR 19:

Average HR during baseline is recorded in register Avg_HR_Baseline 21

HRnu=Current HR−Avg_HR_Baseline

Normalize LF, HF 20:

LFnu=LF/(LF+HF)

HFnu=HF/(LF+HF)

Because HRV varies for many reasons, including personal physiology, age and chronic states of the nervous system, (such as chronic stress, anxiety or depression), the LF and HF values, which are highly representative of the activity in the sympathetic branch of the ANS, are scaled 22. This allows individual who have over active sympathetic activity to still be able to see stress levels that range from low to high. Without this scaling, some people will always show a high level of stress and thus will be unable to gain any benefit from monitoring their stress. Note that the method shown here is adaptive to an individual, meaning that the lowest value of an individual's recorded LFnu is stored in a register MinLFnu 23 and contributes to the extent of the scaling. Therefore an individual with very high LFnu will get more scaling applied than an individual with a lower LFnu. In this embodiment there are 5 levels of scaling or sensitivity where level 1 provides the most scaling and level 5 provides no scaling. The scaling method includes and is not limited to the following:

Sensitivity:

At the end of each session, the following parameter is stored in a register MinLFnu and used for scaling. This allows adaptable scaling for each individual physiology.

a. MinLFnu=Minimum of LFnu of all previous sessions.
i. The default value is 0.6
ii. This register is always updated after the first session
iii. This register will subsequently only be updated if the Min(LFnu) is less than the current register value

Sensitivity scaling for level 1

b. LFnuScaled=LFnu−(MinLFnu −.73)

c. HFnuScaled=LFnu+(MinLFnu −.73)

Sensitivity scaling for level 2

d. LFnuScaled=LFnu−(MinLFnu −.68)

e. HFnuScaled=LFnu+(MinLFnu −.68)

Sensitivity scaling for level 3 (default)

f. LFnuScaled=LFnu−(MinLFnu −.52)

g. HFnuScaled=LFnu+(MinLFnu −.52)

Sensitivity scaling for level 4

h. LFnuScaled=LFnu−(MinLFnu −.48)

i. HFnuScaled=LFnu+(MinLFnu −.48)

Sensitivity scaling for level 5

No Scaling

In order to make rMSSD consumer friendly, it is scaled 24 to a range of 0-100 which is easily understood by most people.

rMSSD Scale:

HRV=(log(rmssd)−0.3)/(2−0.3)*100

HRV will not exceed 100

The normalized heart rate, normalized and scaled LF and HF HRV values are used as the inputs to the stress level classifier 25 that outputs a the detected stress level and measures at the rate of a new value each second.

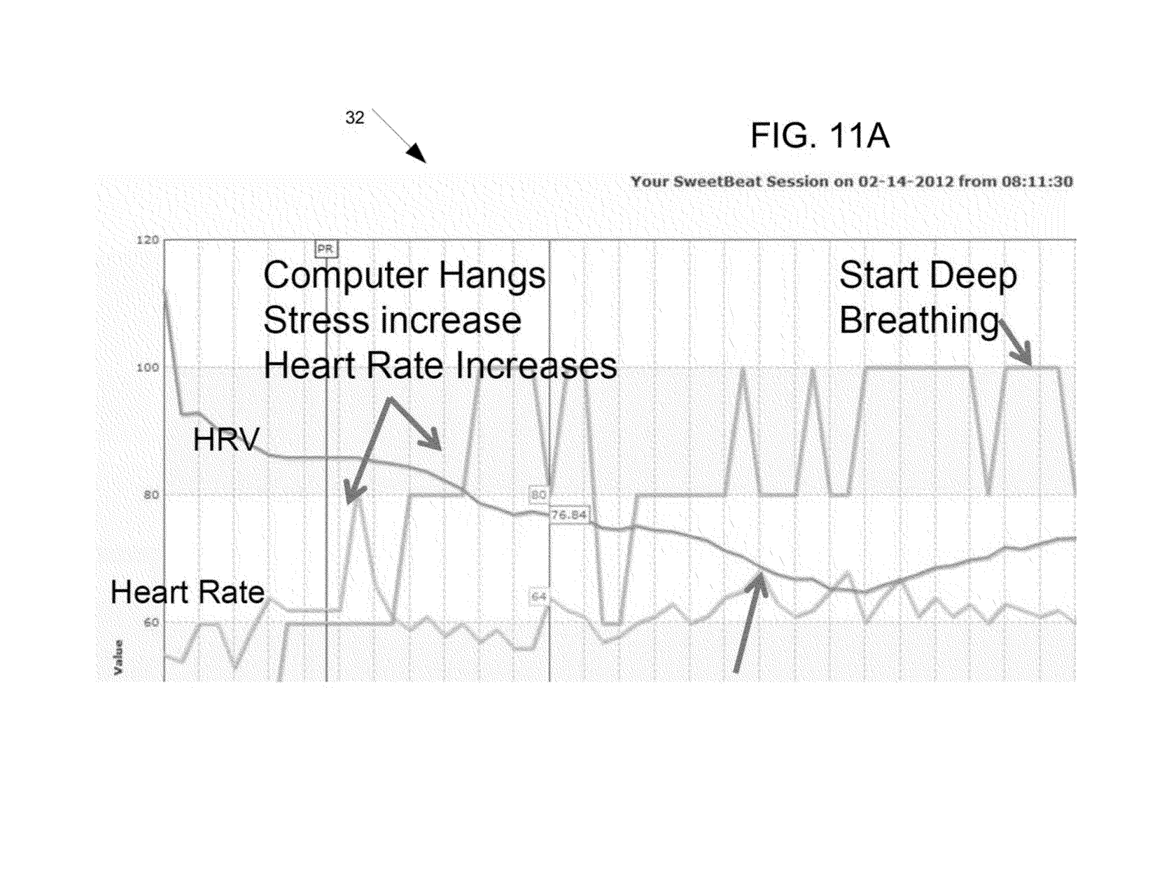

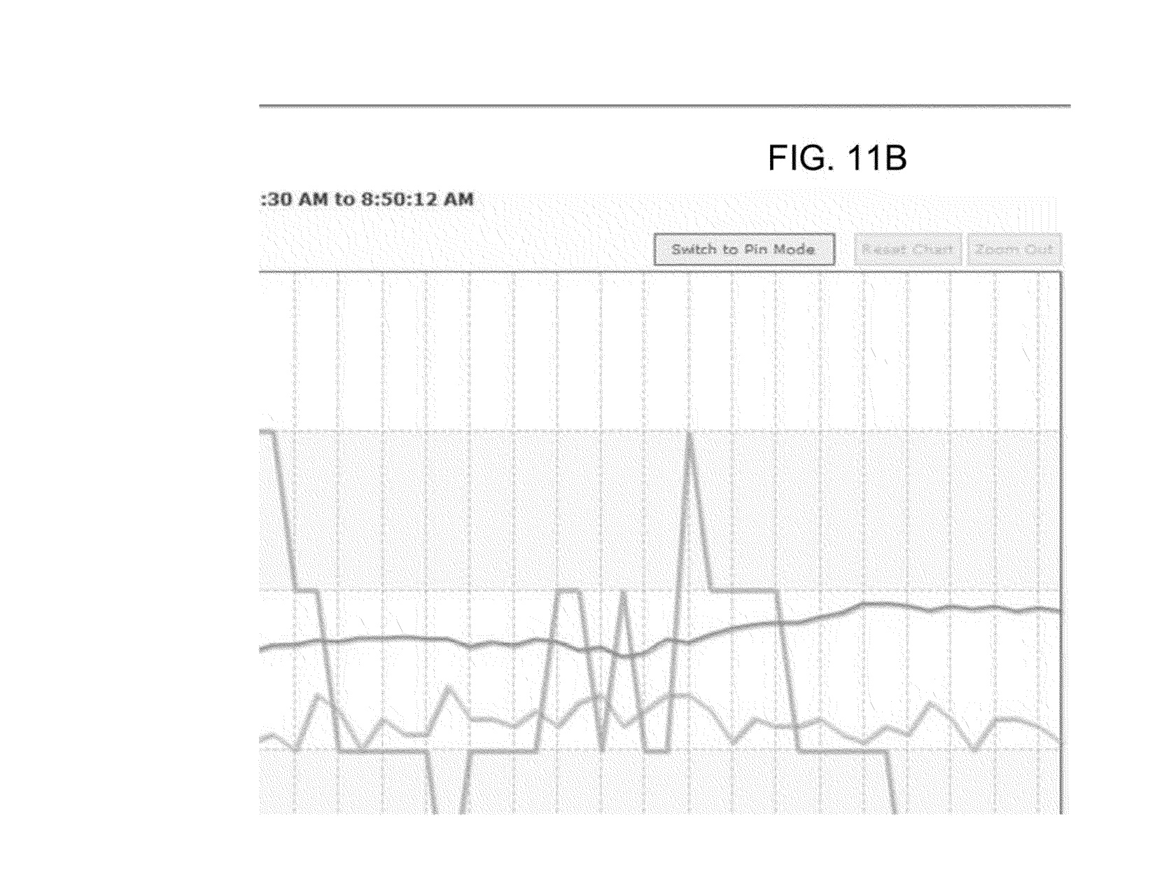

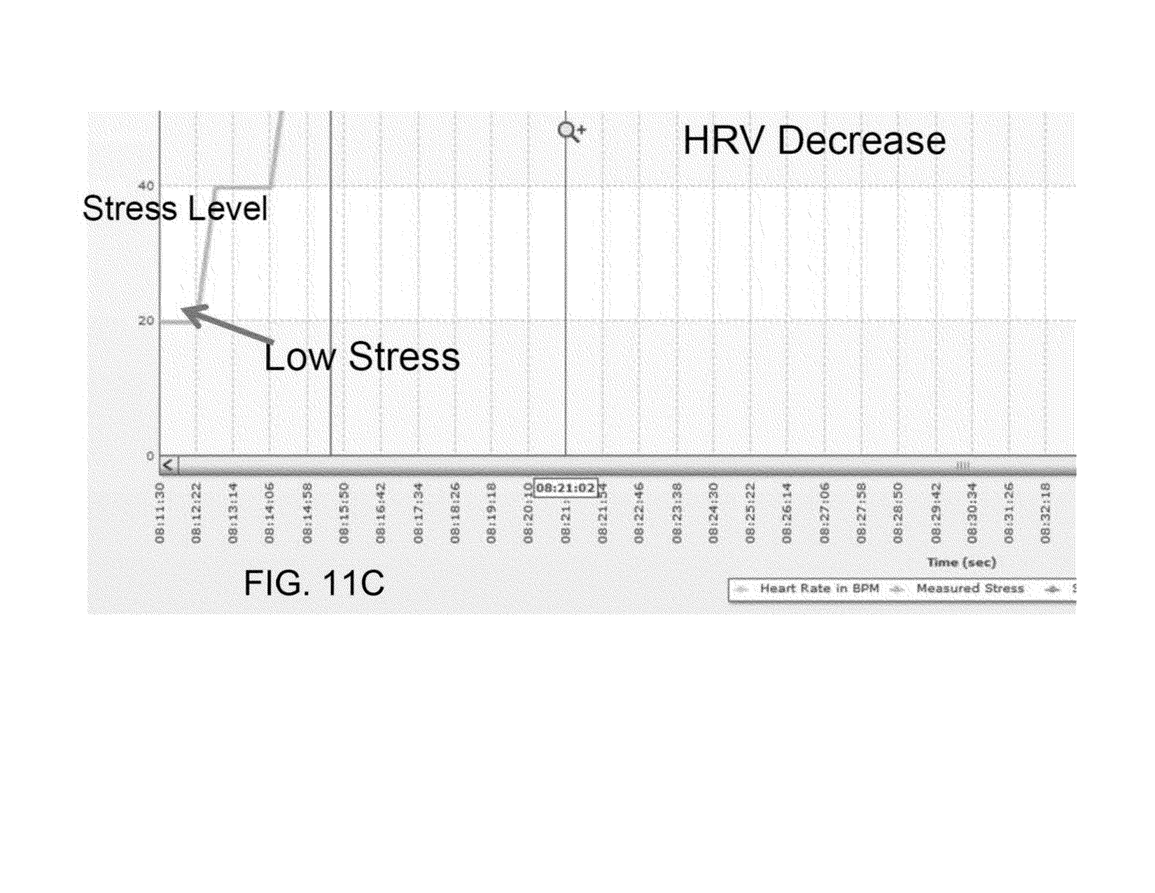

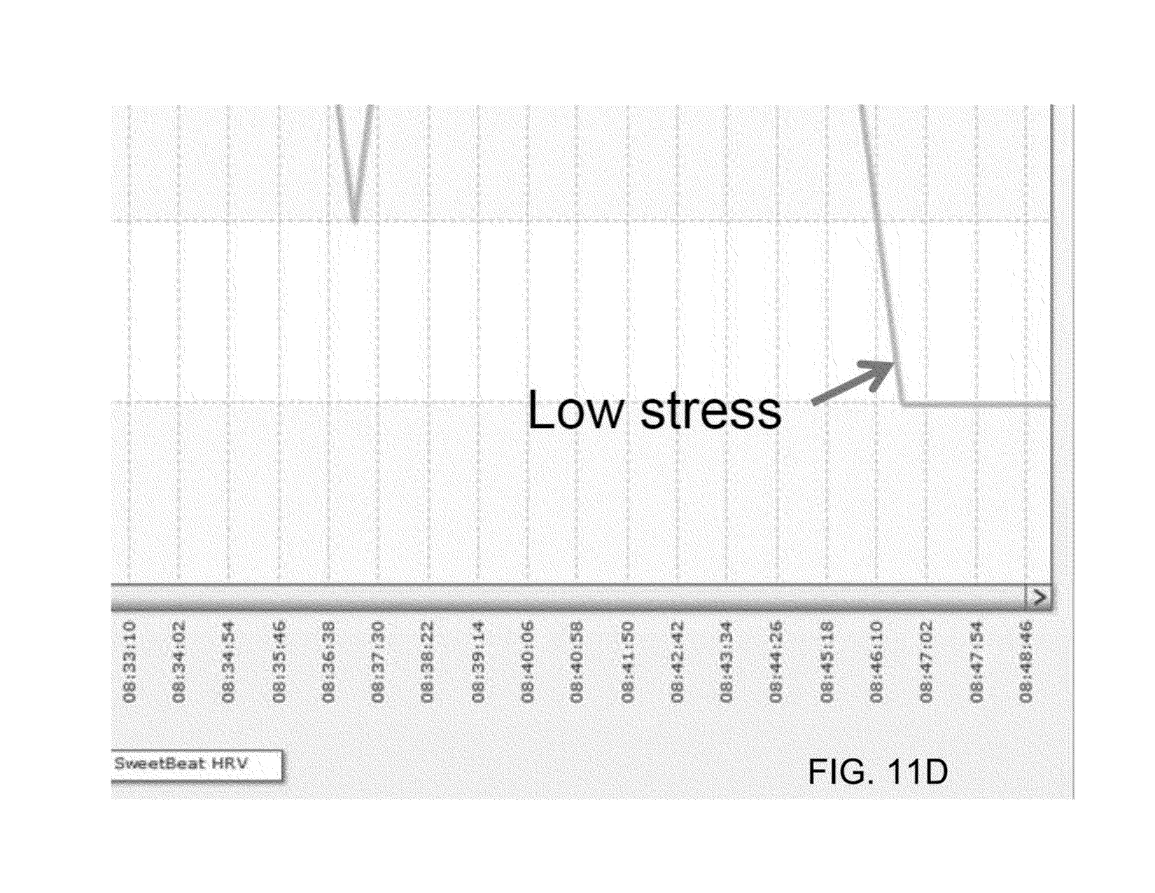

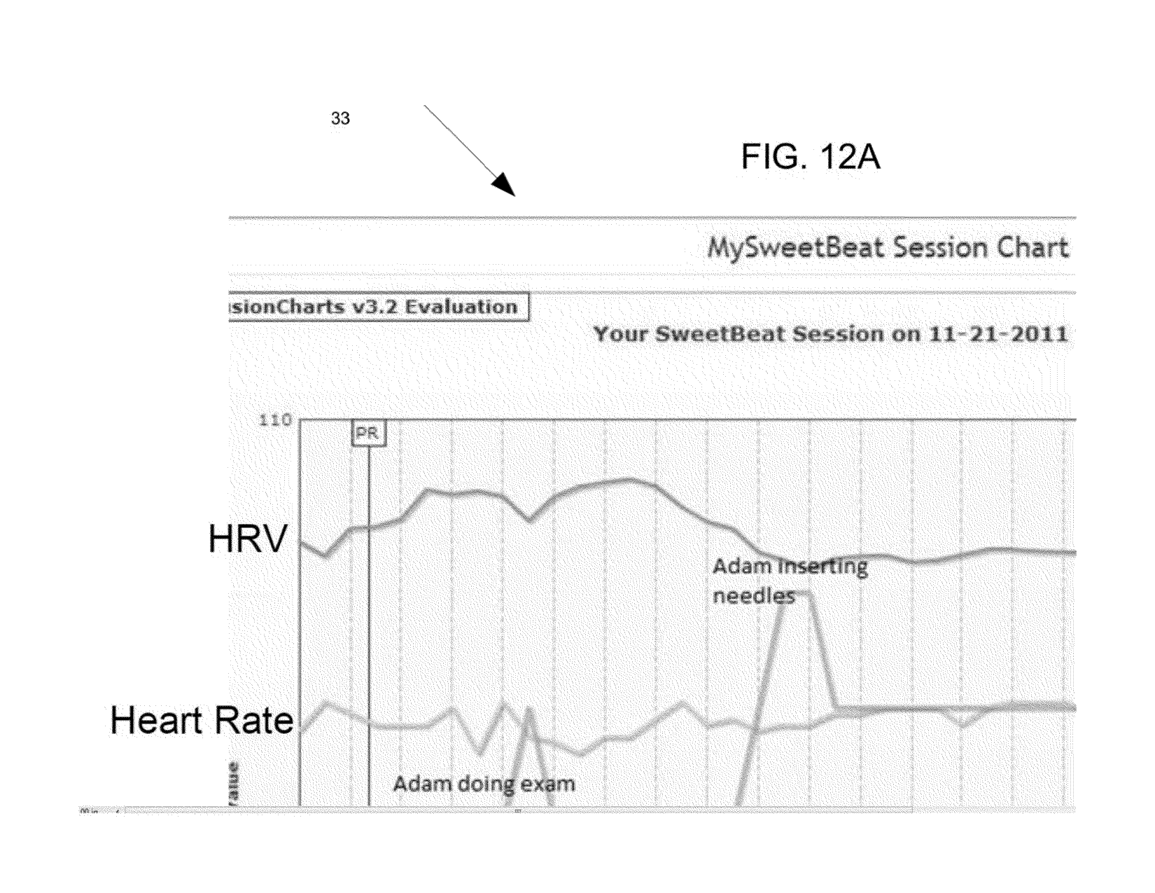

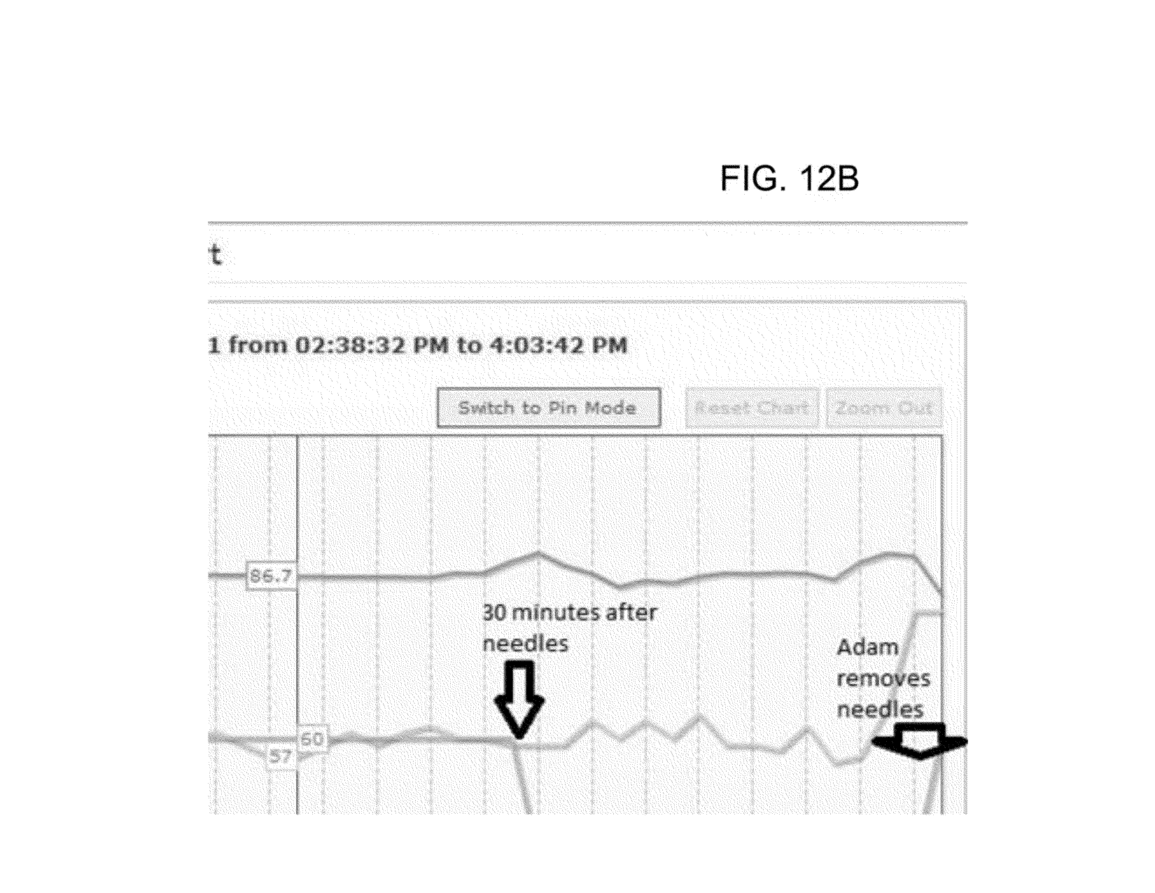

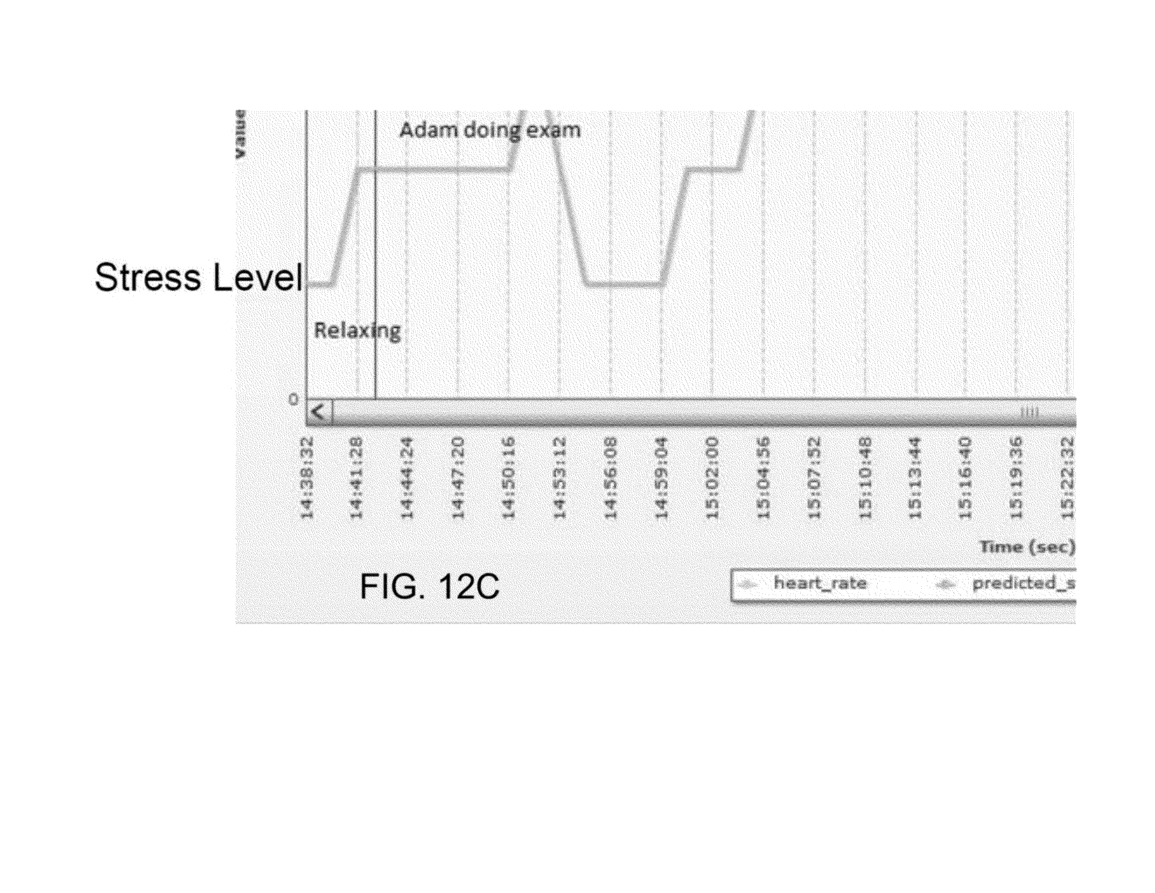

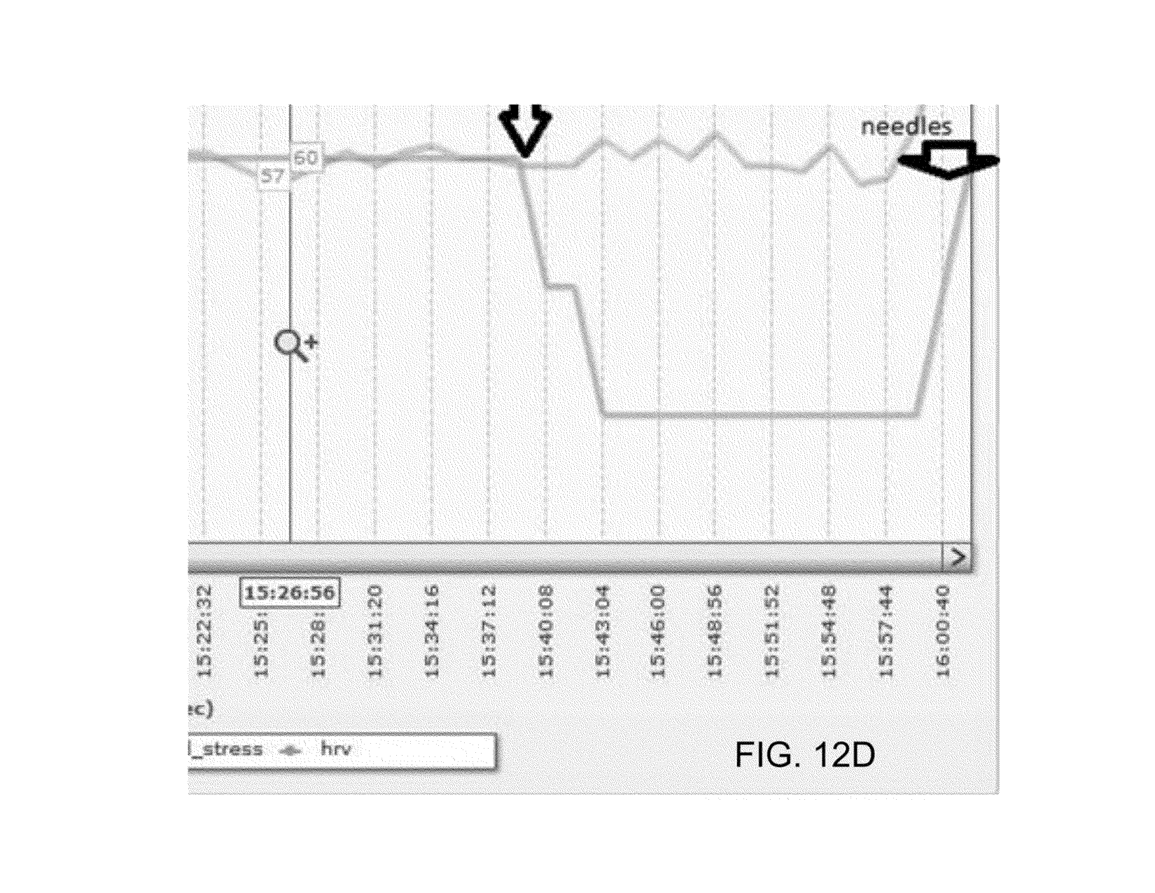

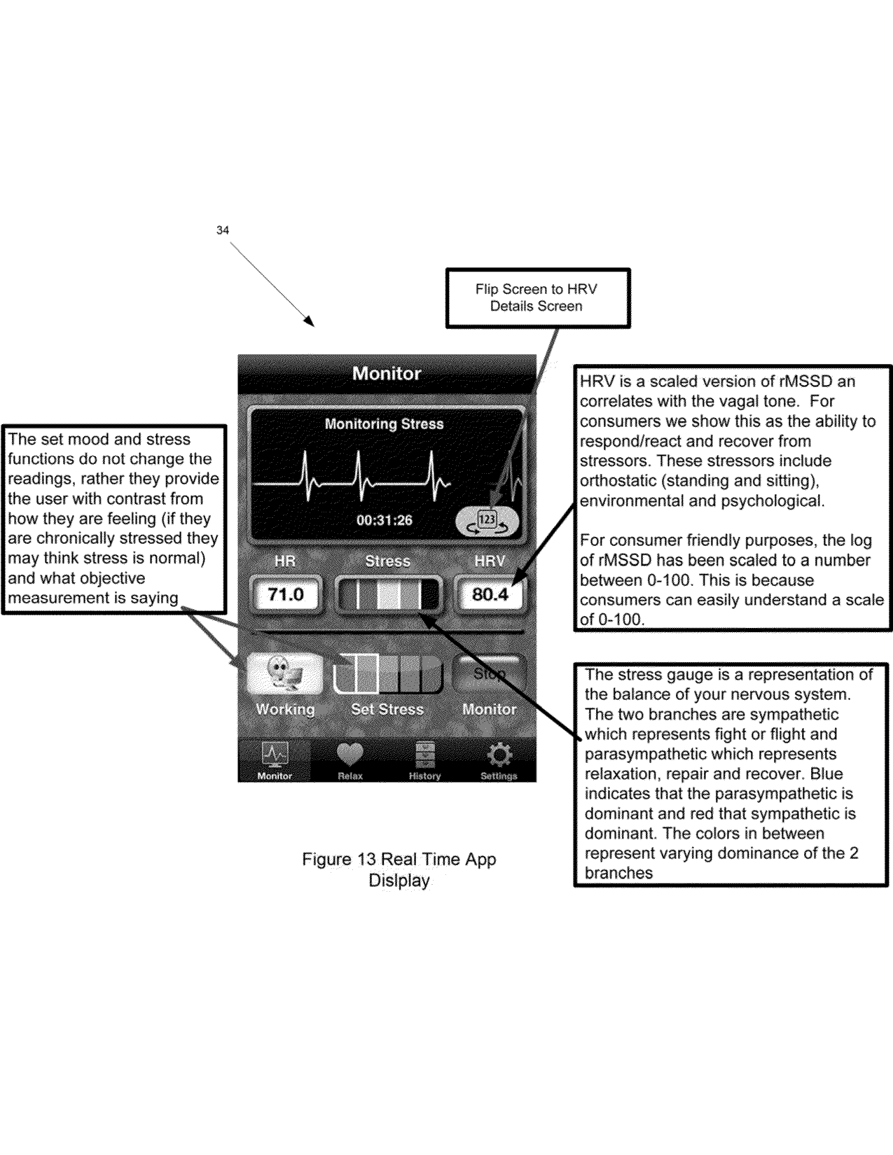

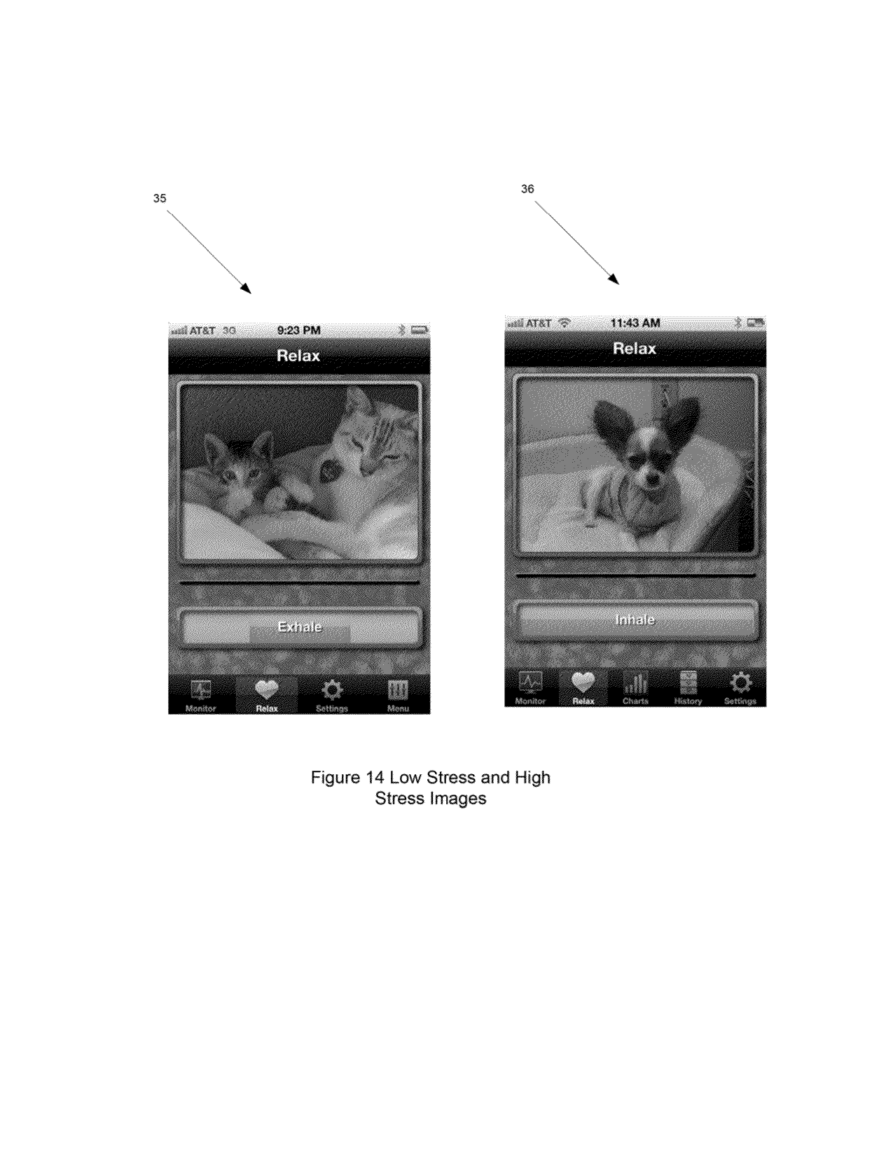

As seen in FIG. 13 the current heart rate, HRV and Stress level are displayed in the application 33 in real time (updated each second) and FIG. 14 shows a visual representation of low 33 and high 34 stress. The real time data, heart rate, HRV and stress levels, are stored and displayed in a graph 31, 32 as shown in FIGS. 11 and 12.

Multilayer Perceptron Detailed Description

The Multilayer Perceptron is a feed forward neural network that maps a set of inputs onto a set of appropriate outputs. The MLP has the following properties:

1 input layer, 3 input nodes (HRnu, LFnuScaled, HFnuScaled)

I Hidden layer

24 nodes in hidden layer

Sigmoid function

1 output layer, 5 output nodes (Stress Level 1-5)

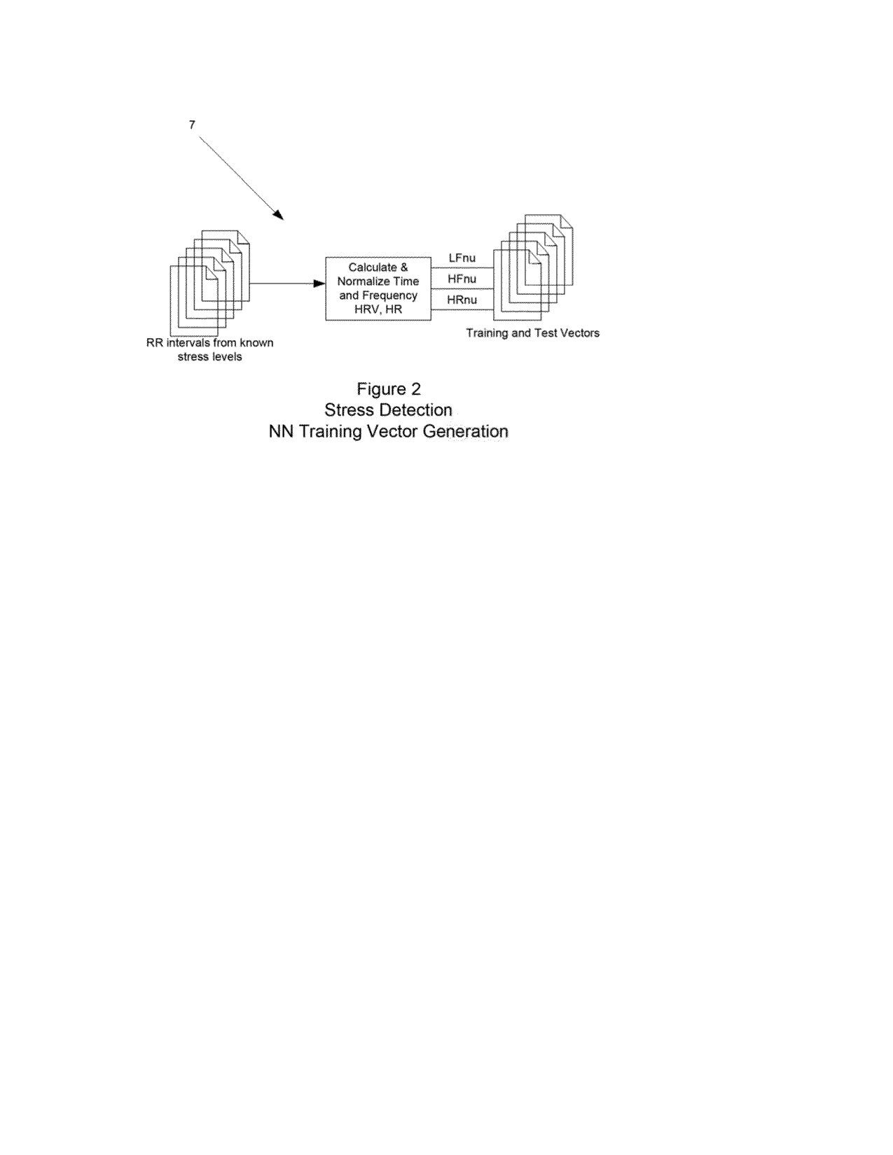

Referring to FIG. 2 the MLP was initially trained using data taken from volunteers while driving on a prescribed route including city streets and. The drivers were presented with the following route, each invoking a range of stress reactions:

Rest in a garage

Drive busy city streets

Drive on the highway

Enter toll booths

The RR intervals and heart rate for low to high stress states were extracted from the data 7 and the HRV calculations were applied to the RR intervals. The resulting HRV and HR were grouped into low, med, medhi, high and highest stress training and test vectors, and applied to training of the MLP.

Once the initial MLP and alpha version of the app was available, more vectors were generated by running sessions in the application in a variety of low to high stress situations, labeling these sessions and combining them into the associated HRV parameters into low-high stress levels. These vectors were combined with the driving training vectors to create a final training and test set.

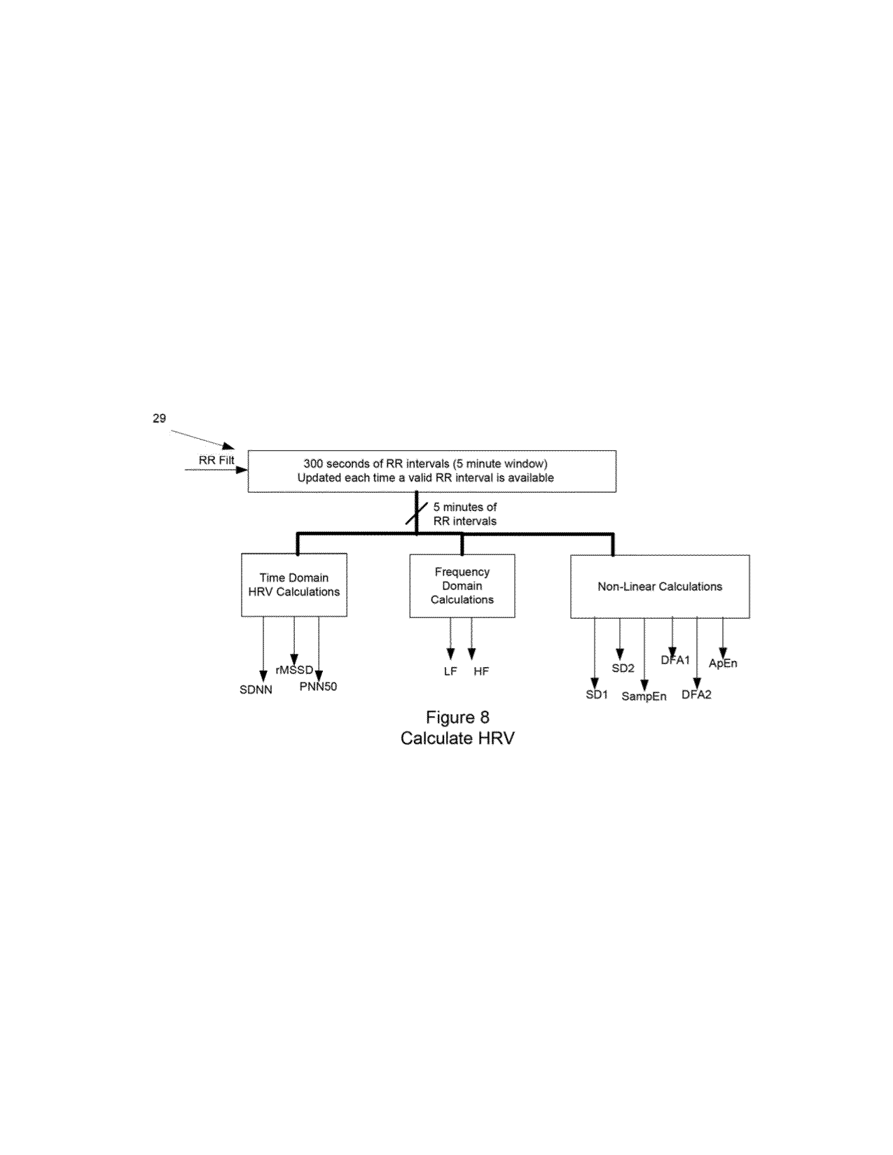

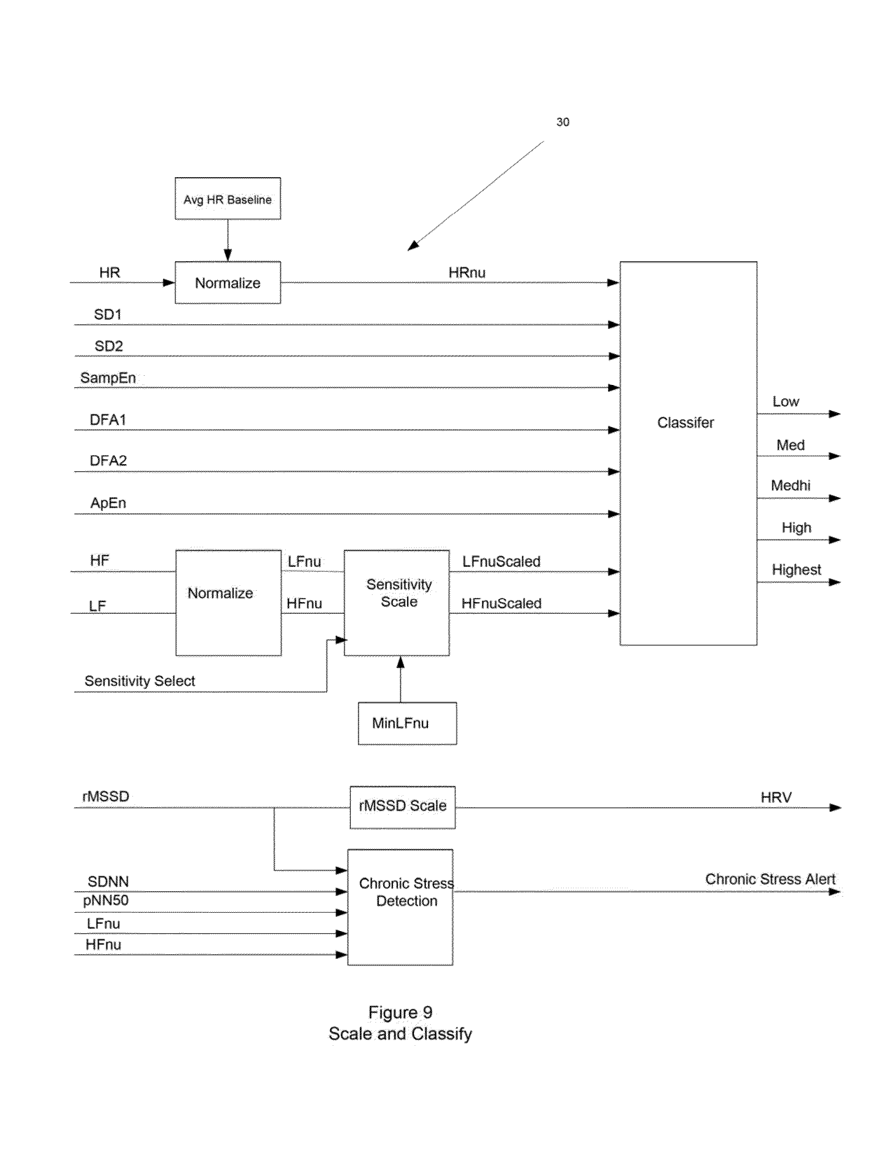

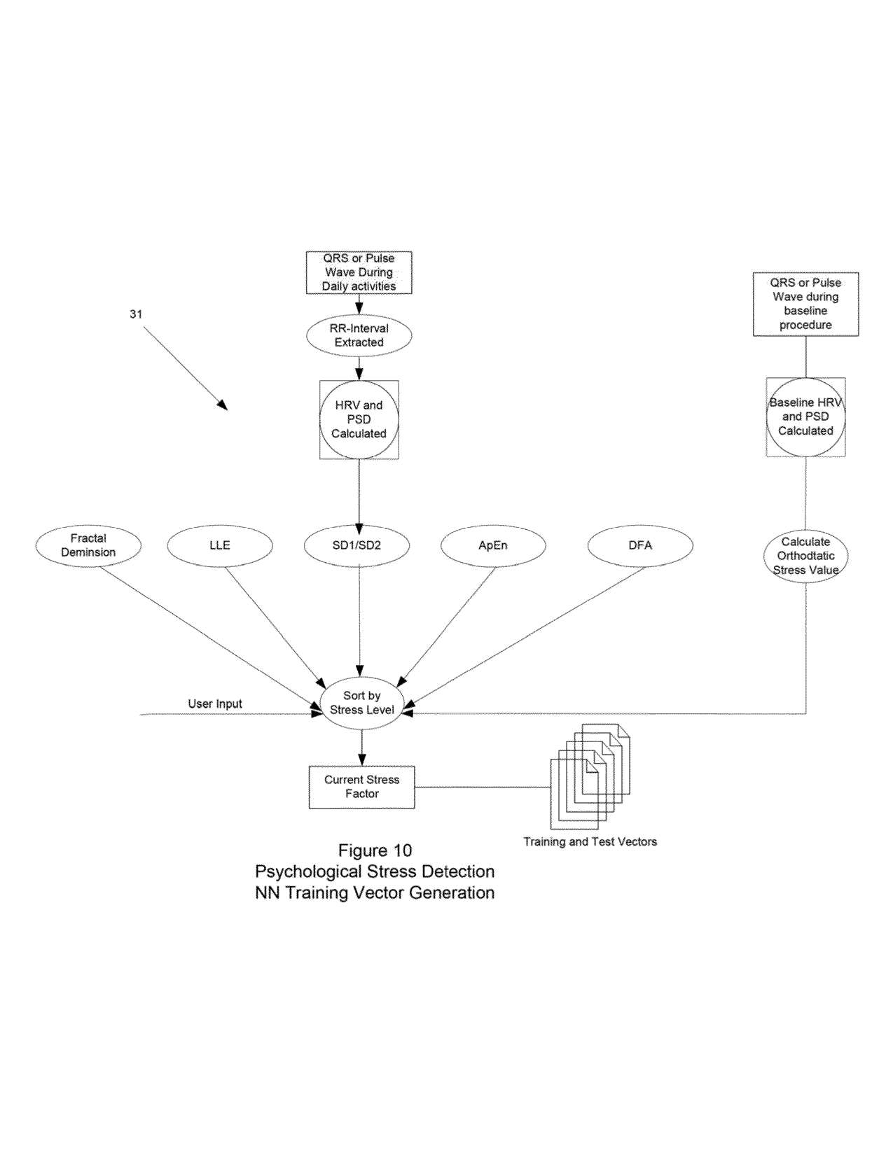

FIGS. 8-10 represent an alternative or complimentary method of stress detection. This method utilizes the non-linear calculations of HRV. Because the RR interval time series of a healthy individual has chaotic and fractal characteristics, the non-linear aspects of HRV can provide deeper insight into the ANS and present an opportunity for early detection and diagnosis for a variety of physical and psychological conditions such as hypertension, heart disease, obstructive sleep apnea, anxiety and depression, to name a few. In addition, burn out or chronic stress can be detected. Tracking these parameters provides individuals and health practitioners a unique insight into the efficacy of treatments.

The methods and systems may be implemented on any computer communicating over any network. For example the computers may include desktop computers, tablets, handheld devices, laptops and mobile devices. The mobile devices may comprise many different types of mobile devices such as cell phones, smart phones, PDAs, portable computers, tablets, and any other type of mobile device operable to transmit and receive electronic messages.

The computer network(s) may include the internet and wireless networks such as a mobile phone network. Any reference to a “computer” is understood to include one or more computers operable to communicate with each other. Computers and devices comprise any type of computer capable of storing computer executable code and executing the computer executable code on a microprocessor, and communicating with the communication network(s). For example computer may be a web server.

References to electronic identifiers may be used which include, but are not limited to, email addresses, mobile phone numbers, user IDs for instant messaging services, user IDs for social networking application or mobile applications, user IDs and URLs for blogs and micro-blogs, URIs, bank account or financial institution numbers, routing numbers, credit and debit cards, any computer readable code, and other electronic identifiers to identify accounts, users, companies, and the like.

The systems and methods may be implemented on an Intel or Intel compatible based computer running a version of the Linux operating system or running a version of Microsoft Windows, Apple OS, and other operating systems. Computing devices based on non-Intel processors, such as ARM devices may be used. Various functions of any server, mobile device or, generally, computer may be implemented in hardware and/or in software, including in one or more signal processing and/or application specific integrated circuits.

The computers and, equivalently, mobile devices may include any and all components of a computer such as storage like memory and magnetic storage, interfaces like network interfaces, and microprocessors. For example, a computer comprises some of all of the following: a processor in communication with a memory interface (which may be included as part of the processor package) and in communication with a peripheral interface (which may also be included as part of the processor package); the memory interface is in communication via one or more buses with a memory (which may be included, in whole or in part, as part of the processor package; the peripheral interface is in communication via one or more buses with an input/output (I/O) subsystem; the I/O subsystem may include, for example, a graphic processor or subsystem in communication with a display such as an LCD display, a touch screen controller in communication with a touch sensitive flat screen display (for example, having one or more display components such as LEDs and LCDs including sub-types of LCDS such as IPS, AMOLED, S-IPS, FFS, and any other type of LCD; the I/O subsystem may include other controllers for other I/O devices such as a keyboard; the peripheral interface may be in communication with either directly or by way of the I/O subsystem with a storage controller in communication with a storage device such a hard drive, non-volatile memory, magnetic storage, optical storage, magneto-optical storage, and any other storage device capable of storing data; the peripheral interface may also be in communication via one or more buses with one or more of a location processor such as a GPS and/or radio triangulation system, a magnetometer, a motion sensor, a light sensor, a proximity sensor, a camera system, wireless communication subsystem(s), and audio subsystems.

A non-transitory computer readable medium, such as the memory and/or the storage device(s) includes/stores computer executable code which when executed by the processor of the computer causes computer to perform a series of steps, processes, or functions. The computer executable code may include, but is not limited to, operating system instructions, communication instruction, GUI (graphical user interface) instructions, sensor processing instructions, phone instructions, electronic messaging instructions, web browsing instructions, media processing instructions, GPS or navigation instructions, camera instructions, magnetometer instructions, calibration instructions, an social networking instructions.

An application programming interface (API) permits the systems and methods to operate with other software platforms such as Salesforce CRM, Google Apps, Facebook, Twitter, social networking sites, desktop and server software, web applications, mobile applications, and the like. For example, an interactive messaging system could interface with CRM software and GOOGLE calendar.

A computer program product may include a non-transitory computer readable medium comprising computer readable code which when executed on the computer causes the computer to perform the methods described herein. Databases may comprise any conventional database such as an Oracle database or an SQL database. Multiple databases may be physically separate, logically separate, or combinations thereof.

The features described can be implemented in any digital electronic circuitry, with a combination of digital and analogy electronic circuitry, in computer hardware, firmware, software, or in combinations thereof. The features can be implemented in a computer program product tangibly embodied in an information carrier (such as a hard drive, solid state drive, flash memory, RAM, ROM, and the like), e.g., in a machine-readable storage device or in a propagated signal, for execution by a programmable processor; and method steps can be performed by a programmable processor executing a program of instructions to perform functions and methods of the described implementations by operating on input data and generating output(s).

The described features can be implemented in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any type of programming language (e.g., Objective-C, Java), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. Some elements of a computer are a processor for executing instructions and one or more memories for storing instructions and data. Generally, a computer will also include, or communicate with one or more mass storage devices for storing data files. Exemplary devices include magnetic disks such as internal hard disks and removable disks, magneto-optical disks, and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) for displaying information to the user and a keyboard and a pointing device such as a mouse or a trackball by which the user can provide input to the computer. The display may be touch sensitive so the user can provide input by touching the screen.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, wired and wireless packetized networks, and the computers and networks forming the Internet.

The foregoing detailed description has discussed only a few of the many forms that this invention can take. It is intended that the foregoing detailed description be understood as an illustration of selected forms that the invention can take and not as a definition of the invention. It is only the claims, including all equivalents, that are intended to define the scope of this invention.

1. A computer program product that as input receives heart beat information from a heart rate monitor and, when run on a computer, processes the heart beat intervals to detect the state of the Autonomic Nervous system in real time using a re-programmable personalized Neural Network, applies adaptive scaling to HRV parameters to customize the detection for individual, and as output generates immediate feedback that includes measured stress levels and, when appropriate, alerts using audio, visual, alphanumeric displays on the computer platform and audio feedback, that may be part of a stress level-audio feedback loop, to a wearable audio transducer.
